(19) United States
(12) Patent Application Publication
(10) Pub. No.: US 2024/0130338 A1
(43) Pub. Date: Apr. 25, 2024

(54) BIRDS FOR PRODUCING FEMALE HATCHLING AND METHODS OF PRODUCING SAME

(71) Applicant: B.G. Negev Technologies and Applications Ltd., at Ben-Gurion University, Beer Sheva (IL)

(72) Inventors: Uri Abdu, Lehavim (IL); Eden Ozer, Rishon Lezion (IL)

(21) Appl. No.: 18/556,761

(22) PCT Filed: Apr. 12, 2022
(86) PCT No.: PCT/IL22/50389

§ 371 Date: Oct. 22, 2023

(30) Foreign Application Priority Data
Apr. 21, 2021 (IL) 282597

(51) Int. Cl. A01K 67/0275; C12N 9/22; C12N 15/10; C12N 15/86

(52) U.S. Cl. CPC A01K 67/0275; C12N 9/22; C12N 15/102; C12N 15/86; A01K 2227/30; C12N 2310/20

(57) ABSTRACT

The present invention provides compositions and methods for generating a genetically modified female bird such that, when crossed with a native male bird, produces selectively only female viable hatched offspring.

### FIELD OF THE INVENTION

The present invention relates to compositions and methods for generating a genetically edited female bird such that, when crossed with a native male bird, produces selectively female, but not male, viable hatched offspring.

### BACKGROUND OF THE INVENTION

In commercial flocks of avian species, particularly chicken, sex separation is an important aspect in the production of broilers (bred and raised for meat production) and egg-laying hens. Sex separation allows a better suited management and feeding according to the breeding line developed to efficiently maximize the end product (meat or eggs). Essentially in all commercial hatcheries billions day-old chicks are culled every year. Males of layer breeds are exterminated since they are not useful and females of broiler breeds are terminated since growing them for meat is not economical.

In avian species sex determination is via female heredity, as Z-Z allosome pair will assign a male and Z-W allosome pair will assign a female (Fridolfsson, A. K. et al. 1998. Proc. Natl. Acad. Sci. U. S. A. 95, 8147-8152). Comparing the avian W chromosome to the human Y chromosome, the two chromosomes conserved minimal identity to ancestral genes, minimizing size and therefore expressed genes. Despite the evolutionary similarities, it was noted that the chicken W chromosome is remarkably divergent from all sequenced Y chromosomes, in that it lacks any genes expressed specifically in sex-specific organs or tissues (Bellott, D. W. et al. 2017. Nat. Genet. 49, 387-394).

Parallel lines of evidence in the chicken lead Bellot et. al (2017, ibid) to propose that the avian sex chromosomes possess a critical combination of genes' expression, ensuring the survival of females. More specifically, the combination of genes ensures a correct embryonic development in early stages.

There is an ongoing search for means and methods for determining the desired sex of an embryo while in the egg. For example, International (PCT) Applications Publication Nos. WO 2017/094015 and WO 2018/216022 discloses non-invasive methods using transgenic avian animals that comprise at least one reporter gene integrated into at least one gender chromosome Z or W. The transgenic avian disclosed therein are used for gender determination and selection of embryos in unhatched avian eggs by detecting the reported gene.

International (PCT) Applications Publication No. WO 2019/092265 discloses a method and an apparatus for automated noninvasive determining the sex of an embryo of a bird's egg, in particular a chicken egg, which allows for a rapid and reliable determination of the sex of the embryo at an early stage, at which the embryo has not developed a sense of pain yet. The method is based on NMR parameters associated with the egg selected from the group consisting of a T1 relaxation time, a T2 relaxation time and a diffusion coefficient, and a classification module configured for determining, based on said one or more NMR parameters or parameters derived therefrom, a prediction of the sex of the embryo of the associated egg.

While avoiding the need to cull living hatchlings, sex sorting of eggs still requires destroying a vast number of eggs comprising living embryos. Attempts have been therefor made to set the offspring sex by manipulating the breeding parents. For example, International (PCT) Application Publication No. WO 2018/013759 discloses a bird or cells thereof comprising an autosomal repressor cassette integrated on at least one copy of an autosome, which can suppress the expression of a protein essential for early development. In some aspects, a bird or cells thereof are provided that comprise an ectopic rescue cassette and a repressor cassette on the W or Z chromosome, which can selectively rescue embryo development in progeny animals. Methods of producing same are also disclosed.

International (PCT) Applications Publication Nos. WO 2019/058376 and WO 2020/178822 disclose DNA editing agents for generating chimeric bird cells and chimeric birds. The agents can be used to produce conditionally-lethal phenotype in male bird embryos. Method for destroying male chick embryos in-ovo are also provided.

US application publication No. 20140359796 discloses genetically modified livestock animals, and methods of making and using the same, the animals comprise a genetic modification to disrupt a target gene selectively involved in gametogenesis, wherein the disruption of the target gene prevents formation of functional gametes of the animal.

However, there is a great need for and would be highly advantageous to have a reproducible and efficient methods for distorting female:male sex ratio in hatchlings of a breeding flock.

### SUMMARY OF THE INVENTION

The present invention answers the above-described needs, providing in some embodiments a genetically modified female bird capable of laying viable egg populations with a sex ratio biased toward females. Advantageously, the female offspring are non-genetically modified. The present invention further provides genetically modified or edited male birds that are used for generating the genetically modified female described herein, and methods for producing a bird hatchling population characterized by a sex ratio biased towards females.

The present invention in based in part on the unexpected discovery that editing at least one Z-chromosome gametolog results in male-only ability to inherit the edited Z-chromosome, while in females, a gamete bearing the edited chromosome, upon fertilization, would not develop into a viable embryo.

Without wishing to be bound by any specific theory or mechanism of action, it is herein disclosed that the non-modified Z chromosome of the male bird compensates and enables the meiosis to produce a gamete having a modified chromosome Z, which may fertilize a female gamete to produce a viable embryo. In contrast, in females having modified Z chromosome, the chromosome W gametolog is not sufficient to enable the generation of a viable male embryo as it requires the product of the Z-gametolog before the fertilization. Advantageously, the methods provided herein enable the production of males that may produce multiple layer females having distorted female: male sex ratio in hatchlings. Methods as described herein utilize a one-step site-directed mutagenesis for the production of birds as described herein, that assure minimal genetic and/or epigenetic adverse effects. The methods described herein in some embodiments, utilize systems that do not integrate any exogenous genes to the genome, and the resulting birds are considered non-transgenic birds.

According to one aspect, the present invention provides a bird cell having at least one genetically modified chromosome Z, wherein the genetically modified chromosome comprises at least one chromosome Z-gametolog having reduced expression and/or activity. In another aspect, the present invention provides a male bird cell having at least one genetically modified chromosome Z, wherein the genetically modified chromosome comprises at least one chromosome Z-gametolog having reduced expression and/or activity, wherein the bird cell is capable of developing into functional gametes

According to some embodiments, the cell is genetically edited using at least one artificially engineered nuclease.

According to some embodiments, the gametolog is a gene selected from the group consisting of zfr, smad2, st8sia3, kcmf1, spin1, sub1, chd1, nipbl, hnrnpk, gfbp1, mier3, btf3, golph3, vcp, txn11, nedd4, ctif, smad7, rpl17, znf532, hintz, c18orf25, atp5a, zswim6, rasa1, ube2r2, ubap2, and tcf4. Each possibility represents a separate embodiment of the invention.

According to some embodiments, the gametolog is genetically modified to reduce its expression. According to some embodiments, the gametolog is genetically modified to reduce its activity.

According to some embodiments, the gametolog is a meiosis-associated gene.

According to some embodiments, the gene is selected from the group consisting of zfr, smad2, spin1, and nipbl.

According to some embodiments, the gene encodes Zinc Finger RNA Binding Protein (ZFR). According to certain embodiments, the gene is zfr.

According to some embodiments, the cell is a primordial germ cell (PGC). According to some embodiments, the PGC is selected from the group consisting of gonadal PGC, blood PGC and germinal crescent PGC. In another embodiment, the cell is a spermatogonial stem cell (SSC). In other embodiments, the cell is a spermatogonium or a spermatocyte. In another embodiment, the cell is a gamete (e.g. sperm cell).

According to some embodiments, when the bird is a male, the cell is heterozygous to the genetically edited chromosome Z.

According to some embodiments, the bird is a poultry. According to some embodiments, the bird is selected from the group consisting of chicken, quail, turkey, goose, and duck. According to certain embodiments, the bird is a chicken or quail. According to additional embodiments, the bird is an ornamental bird.

According to some embodiments, there is provided a cell population comprising the at least one cell. According to some embodiments, the cell population comprises gametes.

According to some embodiments, a bird having the at least one cell is provided. According to certain embodiments, the bird is a non-transgenic bird.

According to some embodiments, the bird is a chimeric bird. According to certain embodiments, the bird is a chimeric male bird having at least one PGC as described herein. According to certain embodiments, the at least one PGC is heterozygous to the genetically edited chromosome Z.

According to some embodiments, the bird is a female bird. According to some embodiments, the bird is a female bird having at least one PGC as described herein.

According to an additional aspect, the present invention provides a site-directed mutagenesis system for reducing the expression and/or activity of at least one chromosome Z-gametolog.

According to some embodiments, the site-directed mutagenesis system is Clustered Regularly Interspaced Short Palindromic Repeats (CRISPR). According to other embodiments, the site directed mutagenesis comprises the use of zinc-finger nucleases (ZFNs) or transcription activator-like effector nucleases (TALENs).

According to an additional aspect, the present invention provides a gene-editing agent comprising a nucleotide sequence hybridizable with a target nucleic acid sequence within a bird chromosome Z-gametolog.

According to some embodiments, the gene-editing agent is a synthetic guide RNA (sgRNA).

According to some embodiments, the sgRNA comprises a nucleotide sequence complementary to a target nucleic acid sequence within a bird chromosome Z-gametolog. In particular, provided is a sgRNA comprising a targeting sequence (crRNA) comprising 15-30 contiguous nucleotides that are specifically hybridizable (hybridizes, or is capable of hybridizing, in a selective manner) with a target nucleic acid sequence within a bird chromosome Z-gametolog.

According to some embodiments, the targeting sequence (crRNA) is at least 90%, at least 95% or at least 98% complementary to a target nucleic acid sequence within a bird chromosome Z-gametolog.

According to some embodiments, the targeting sequence is fully complementary to a target nucleic acid sequence within a bird chromosome Z-gametolog.

According to some embodiments, the target nucleic acid sequence is within the coding region of the gametolog. In other embodiments, the target nucleic acid sequence is within the non-coding region of the gametolog.

According to some embodiments, the Z-gametolog is a gene selected from the group consisting of zfr, smad2, st8sia3, kcmf1, spin1, sub1, chd1, nipbl, hnrnpk, gfbp1, mier3, btf3, golph3, vcp, txn11, nedd4, ctif, smad7, rpl17, znf532, hintz, c18orf25, atp5a, zswim6, rasa1, ube2r2, ubap2, and tcf4. Each possibility represents a separate embodiment of the invention.

According to some embodiments, the gametolog is a meiosis-associated gene.

According to some embodiments, the gene is selected from the group consisting of zfr, smad2, spin1, and nipbl.

According to some embodiments, the gene encodes a zinc finger RNA binding protein (ZFR).

According to some embodiments, the target nucleic acid sequence is within exon 3 of zfr.

According to some embodiments, the synthetic guide RNA comprises a targeting sequence selected from the group consisting of GGCTAGCTACACTGTCCACC (SEQ ID NO: 1) and GCGCACACAGCTACAGATTA (SEQ ID NO: 2).

According to some embodiments, a nucleic acid construct encoding the synthetic guide RNA is provided.

According to some embodiments, a vector comprising at least one nucleic acid as described herein is provided. According to certain embodiments, the vector is a viral vector. According to certain embodiments, the viral vector is of a lentivirus or adenovirus. In a particular embodiment said vector is a lentivirus.

According to some embodiments, the bird is poultry. According to certain embodiments, the bird is a chicken or quail.

According to an aspect, the present invention provides an engineered, non-naturally occurring Clustered Regularly Interspaced Short Palindromic Repeats (CRISPR) gene-editing system comprising: (i) a synthetic guide RNA as described herein; and (ii) an RNA-guided DNA endonuclease enzyme.

According to some embodiments, the endonuclease is selected from the group consisting of caspase 9 (Cas9), Cpf1, zinc-finger nucleases (ZFNs), and transcription activator-like effector nucleases (TALENs).

According to some embodiments, the CRISPR editing system comprises a first nucleic acid sequence encoding the synthetic guide RNA and a second nucleic acid sequence encoding the RNA-guided DNA endonuclease enzyme. According to certain embodiments, the first and the second nucleic acid sequences each form a separate molecule. According to additional embodiments, the first and the second nucleic acid sequences are comprised in a single molecule.

According to some embodiments, a vector comprising the at least one engineered non-naturally occurring gene-editing system is provided. According to some embodiments, the vector is a viral vector. According to certain exemplary embodiments, the viral vector is of lentivirus or adenovirus. In a particular embodiment said vector is a lentivirus.

According to some embodiments, a cell population comprising the gene-editing system is provided.

According to some embodiments, the genetically modifying or editing system is transiently expressed in the cells.

According to some embodiments, a bird (e.g. male) comprising at least one cell comprising the gene-editing system is provided. According to certain embodiments, the at least one cell is a PGC. In another embodiment, the cell is selected from the group consisting of gonadal PGC, blood PGC and germinal crescent PGC. In another embodiment, the cell is a spermatogonial stem cell (SSC). In other embodiments, the cell is a spermatogonium or a spermatocyte. In another embodiment, the cell is a gamete (e.g. sperm cell).

According to an additional aspect, the present invention provides a chimeric male bird having cells with a genetically modified chromosome Z comprising at least one chromosome Z-gametolog having reduced expression and/or activity and an unmodified chromosome Z.

According to some embodiments, the cells are genetically edited using at least one artificially engineered nuclease.

According to some embodiments, the bird does not comprise any exogenous polynucleotide sequence stably integrated into its genome. According to certain embodiments, the bird does not comprise the genetically modifying or gene editing system described herein. According to other embodiments, the bird comprises an exogenous polynucleotide sequence stably integrated into its genome.

According to an aspect, the present invention provides a method of generating a chimeric male bird having cells with a genetically modified chromosome Z comprising at least one chromosome Z-gametolog having reduced expression and/or activity and an unmodified chromosome Z, the method comprising the step of applying the site-directed mutagenesis system or the gene-editing system as described herein to a population of male bird cells, thereby generating genome-modified bird cells; and transferring the genome-modified bird cells to a recipient male bird embryo, thereby generating the chimeric male bird.

According to some embodiments, the method comprises a step of abolishing or disrupting the endogenous PGCs cells of the recipient bird before transferring the genome-modified bird cells to the recipient bird.

According to some embodiments, the method comprises raising the chimeric bird to sexual maturity, wherein the chimeric bird produces gametes derived from the donor PGCs.

According to an aspect, the present invention provides a method of generating a chimeric male bird having cells with a genetically modified chromosome Z, the cells comprising at least one chromosome Z-gametolog having reduced expression and/or activity and an unmodified chromosome Z, the method comprising the step of administering the site-directed mutagenesis system or the gene-editing system as described herein to a recipient male bird embryo.

According to some embodiments, the site-directed mutagenesis system or the gene-editing system are administered via a route selected from the group consisting of a viral infection, transposase system, electroporation, chemical transformation, or any combination thereof. According to exemplary embodiments, the viral infection is by a lentivirus or adenovirus.

According to an additional aspect, the present invention provides a method of generating a chimeric male bird having cells with a genetically modified chromosome Z, the cells comprising at least one chromosome Z-gametolog having reduced expression and/or activity and an unmodified chromosome Z, the method comprising the step of administering the site-directed mutagenesis system or the gene-editing system as described herein in-vivo to a recipient male bird.

According to some embodiments, the bird is a sexually mature male bird.

In various embodiments, the site-directed mutagenesis system or the gene-editing system may be administered directly to gametes and/or precursors thereof (e.g. SSC or other spermatogonia) of a male bird in vivo. According to some embodiments, the site-directed mutagenesis system or the gene-editing system are administered directly to the male bird testicles (e.g. by intra-testicular injection). According to some embodiments, the site-directed mutagenesis system or the gene-editing system is administered via a route selected from the group consisting of a viral infection, transposase system, electroporation, chemical transformation, or any combination thereof.

According to certain embodiments, the site-directed mutagenesis system or the gene-editing system is administered using lentivirus.

The bird, gametolog gene and the site-directed mutagenesis system or the gene-editing system are as described hereinabove.

According to another aspect, the present invention provides a method of generating a chimeric male bird having cells with a genetically modified chromosome Z, the cells comprising at least one chromosome Z-gametolog having reduced expression and/or activity and an unmodified chromosome Z, the method comprising the step of administering the genetically modified PGCs as described herein to a recipient male bird.

According to some embodiments, the bird is sexually mature male bird. According to certain embodiments, the method comprises a step of administering the cells to the bird testicles. According to certain embodiments, the bird was sterilized before the administering of the genetically modified PGCs.

According to an additional aspect the present invention provides a genetically modified male bird comprising at least one cell comprising genetically modified chromosome Z comprising at least one chromosome Z-gametolog having reduced expression and/or activity and an unmodified chromosome Z.

According to some embodiments, there is provided a method for generating the genetically modified male bird comprising the step of mating a chimeric male bird as described herein with a female bird having unmodified chromosome Z, and screening the resulting offspring for genetically modified males.

According to an additional aspect, the present invention provides a genetically modified female bird capable of laying viable egg population with biased sex ratio, said bird having a reduced expression and/or activity of at least one chromosome Z-gametolog.

According to some embodiments, there is provided a method for generating the genetically modified female bird capable of laying viable egg population with biased sex ratio, comprising the step of crossing the genetically modified male bird described herein with a female bird and screening the offspring for genetically modified females.

According to an additional aspect, the present invention provides a method for producing a bird hatchling population characterized by a sex ratio biased towards females, comprising breeding the genetically modified female bird as described herein with a male bird having unmodified Z-chromosome, thereby producing an essentially female-only hatchling population.

According to an additional aspect, the present invention provides a bird cell having at least one genetically modified chromosome Z, wherein the genetically modified chromosome comprises at least one chromosome Z-gametolog having reduced expression and/or activity.

According to some embodiments, the bird cell is capable of developing into functional gametes.

It is to be understood that any combination of each of the aspects and the embodiments disclosed herein is explicitly encompassed within the disclosure of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1A schematic representation of the breeding steps for generating the genetically modified birds according to some embodiments of the invention and the non-modified female offspring. A) Generation of a ZZ* chimera male. B) Chimera male ZZ* from step A is mated with native females WZ followed by screening for heterozygote male ZZ* offspring. C) Heterozygote male ZZ* from step B is mated with native female WZ followed by screening for heterozygote female WZ* offspring. D) The heterozygote female WZ* from step C is mated with native male ZZ and produces only WZ offspring.

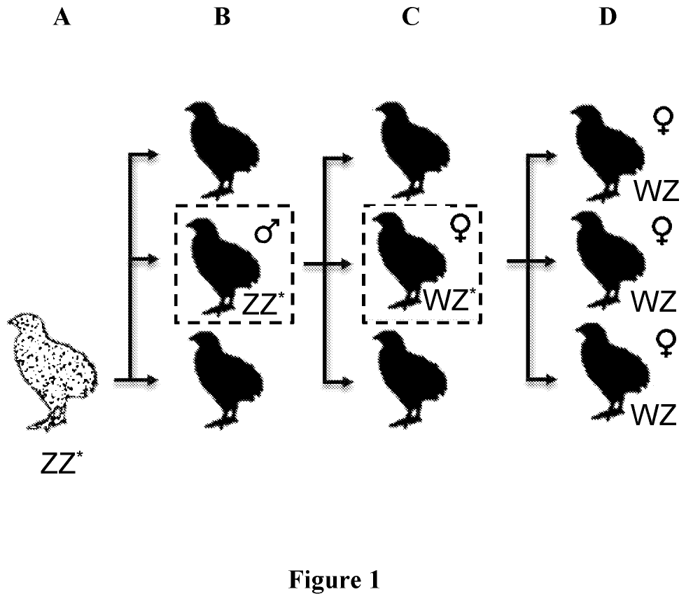

FIG. 2. Agarose analysis for in-vitro cleavage using the gRNA/Cas9 system as described herein. Control lane contained 250 ng of the non-digested target DNA sequence. gRNA lanes were the product of Cas9 endonuclease activity on 250 ng target DNA sequence with gRNA 1 (having SEQ ID NO: 1) or 3 (having SEQ ID NO: 2), marked on the gel, respectively.

FIG. 3. Iv-vivo cleavage assay. a) A schematic representation of the experimental procedure. Genetic construct possessing EGFP gene with a desired genetic target “break” in the middle. EGFP has the potential of assembling a functional EGFP gene if desired genetic target is cleaved. b) Bright field merged with 488 nm channel of HEK cells 72 hrs after co-transfection. c) Bright field merged with 488 nm channel of DF-1 cells 72 hrs after co-transfection. All cells were co-transfected with the 1^{st} plasmid harboring pEGxxFP zfr construct and the 2^{nd} plasmid harboring gRNA (apart from the control experiment, without gRNA) and Cas9 endonuclease. gRNA 1—Guide RNA comprising SEQ ID NO: 1, gRNA 3—Guide RNA comprising SEQ ID NO: 2.

### DETAILED DESCRIPTION OF THE INVENTION

The present invention provides genetically edited birds that produce selectively hatched female offspring. The present invention further provides methods for producing the genetically edited female birds. The present invention further provides genetically edited male birds having cells with a genetically edited chromosome Z comprising at least one chromosome Z-gametolog having reduced expression and/or activity and an unmodified chromosome Z. The genetically edited male birds can be mated with females to result with the genetically modified female birds.

Commercial hatcheries use sex separation during the cultivation of broilers and egg layers. To produce egg laying hens, male chicks are typically culled at the hatchery. The present invention in embodiments thereof provides methods to produce female birds (e.g. chickens) that lay essentially only female offspring. This prevents the inhumane killing of the male chicks and has the economic advantages of reducing feed and energy costs, saving space and manpower.

Methods in accordance of the present invention involve the editing of Z-chromosome gametolog which results in a male-only ability to inherit the edited Z-chromosome. The male gamete having the modified chromosome Z upon fertilization with a native female will develop to a viable embryo. The male birds of embodiments of the invention, having a gamete with a modified chromosome Z-gametolog, can be mated with female birds to produce layer females that can only hatch females. Advantageously, a single male edited in its chromosome Z-gametolog, when mated with females, can produce multiple females, each laying only females.

The present invention discloses for the first time a chromosome Z gametolog, which its function is reduced or abolished at a targeted time after meiosis and until a few days after fertilization in females, results in non-viable male embryo. Without wishing to be bound by any specific theory or mechanism of action, this phenomenon may be attributed to the fact that in females both chromosomes Z- and W-functional gametologs are required for producing a viable embryo. The female gamete requires specific conditions and expression profile prior to fertilization, which later are being used for fertilization and also post-fertilization for the establishment of the embryo in its first days. The male embryo does not survive more than a few days due to lack of the Z-gametolog product. Accordingly, the invention in embodiments thereof provides methods and means for producing heterozygous male birds capable of mating with female birds to produce layer females that can only hatch females.

The present invention provides in some embodiments methods that utilize site-directed mutagenesis for disrupting the expression or activity of a chromosome Z-gametolog in primordial germ cells (PGCs). The genetically modified PGCs are administered in some embodiments to a male embryo to generate a chimeric male having ZZ* (Z* represents a Z chromosome having a genetically modified gametolog). This chimeric male bird, when crossed with a native female bird, enables the generation of a male bird that is heterozygous to the Z gametolog (ZZ*). The heterologous male bird is then breed with a female bird for generating female birds having modified chromosome Z-gametolog (WZ* birds) that are capable of laying only viable female offspring.

In other embodiments, the site-directed mutagenesis is applied directly to testicles of a sexually mature male bird to thereby disrupt the expression or activity of the chromosome Z-gametolog in sperm cells and/or precursors thereof. In some embodiments, viral vectors are used to deliver the site-directed mutagenesis system to the bird testicles.

In additional embodiments, the genetically modified PGCs may be administered to (grafted into) testicles of a sexually mature male bird. In some embodiments, the bird is sterilized prior to the PGCs administration.

According to one aspect, the present invention provides a bird cell having at least one genetically modified chromosome Z, wherein the genetically modified chromosome comprises at least one chromosome Z-gametolog having reduced expression and/or activity. According to another aspect, the present invention provides a male bird cell having at least one genetically modified chromosome Z, wherein the genetically modified chromosome comprises at least one chromosome Z-gametolog having reduced expression and/or activity, the bird cell is capable of developing into functional gametes.

As used herein, the term “genetically modified” with reference to a cell or an organism refers to a cell genetically altered by man or an organism comprising same. The genetic modification includes a modification of an endogenous DNA molecule(s) or gene(s) for example by introducing insertion, alteration, deletion transposable element and the like into an endogenous nucleic acid sequences or gene of interest. Additionally, or alternatively, genetic modification includes transforming the cell with heterologous polynucleotide that incorporate to the cell genome, thereby producing a transgenic cell or a transgenic organism comprising same.

The term “native bird” as used herein refers to a bird that is non-edited or modified in its sex chromosome according to the invention.

The term “chimeric bird” as used herein refers to a bird having both non-edited or modified cells, and modified or edited cells as described herein (i.e. having a genetically modified chromosome Z in which at least one gametolog has reduced expression and/or activity).

According to an aspect the present invention provides a genetically modified male bird comprising at least one cell comprising genetically modified chromosome Z comprising at least one chromosome Z-gametolog having reduced expression and/or activity and an unmodified chromosome Z.

In another embodiment, the present invention provides a genetically modified bird (e.g. male bird) bird comprising, in substantially all its cells, a genetically modified chromosome Z comprising at least one chromosome Z-gametolog having reduced expression and/or activity and an unmodified chromosome Z.

In another embodiment, the present invention provides a genetically modified bird (e.g. male bird) in which the germline cells comprise a genetically modified chromosome Z comprising at least one chromosome Z-gametolog having reduced expression and/or activity and an unmodified chromosome Z.

As used herein a “genetically modified bird” generally refers to a bird in which its cells comprising genetically modified chromosome Z. This term includes a bird in which substantially portion of its cells are modified as described herein. In other embodiments, all of the bird's cells are modified as described herein.

The terms “reduced expression” or “inhibited expression” of a gametolog as described herein are used interchangeably and include, but are not limited to, deleting or disrupting the gene that encodes for the protein to result in a significantly downregulated expression.

The terms “reduced activity” or “inhibited activity” of a gametolog as described herein includes without limitation mutations or posttranslational modifications resulting in a significantly reduced or abolished activity of the protein.

According to some embodiments, the expression or the activity of the gametolog is reduced by at least 50%, 60%, 80%, 80%, 90%, 95%, or 99% compared to the expression or activity of a non-edited or non-modified gametolog. According to some embodiments, the expression of the gametolog is completely abolished. According to additional embodiments, the activity of the gametolog is completely abolished.

The term “functional gamete” as used herein refers to a gamete that is capable, when combined with another male or female gamete, to produce a viable embryo.

A “viable embryo” refers to an embryo that is capable to develop to a bird.

According to some embodiments, an endogenous gene of a cell is modified by gene edited techniques using at least one artificially engineered nuclease.

RNA-directed DNA nucleases are used herein to introduce a mutation(s) in a chromosome Z-gametolog to disrupt its activity and/or expression.

As used herein the term “genetically edited” refers to the insertion, deletion or replacement of one or more nucleotides in endogenous genomic DNA. The insertion, deletion, or replacement are used herein to disrupt the expression and/or activity of a gene product.

The term “gametolog” as used herein is as known in the art and refers to the homologous genes shared between the sex chromosomes, specifically chromosome Z and chromosome W of birds.

According to some embodiments, the gametolog is a gene selected from the group consisting of zfr, smad2, st8sia3, kcmf1, spin1, sub1, chd1, nipbl, hnrnpk, gfbp1, mier3, btf3, golph3, vcp, txn11, nedd4, ctif, smad7, rpl17, znf532, hintz, c18orf25, atp5a, zswim6, rasa1, ube2r2, ubap2, and tcf4. Each possibility represents a separate embodiment of the invention.

According to some embodiments, the gametolog is a gene selected from the group consisting of zfr, smad2, st8sia3, kcmf1, spin1, sub1, chd1, and nipbl. According to some embodiments, the gametolog is a gene selected from the group consisting of hnrnpk, gfbp1, mier3, btf3, golph3, vcp, txn11, nedd4, ctif, smad7, rpl17, znf532, hintz, c18orf25, atp5a, zswim6, rasa1, ube2r2, ubap2, and tcf4. Each possibility represents a separate embodiment of the invention.

According to some embodiments, the at least one gametolog is genetically modified to reduce its expression. According to some embodiments, the at least one gametolog is genetically modified to reduce its activity. The modification can be done, for example, by the insertion of a missense or nonsense mutation to the coding region.

According to some embodiments, the gametolog is a meiosis-associated gene.

According to some embodiments, the gene is selected from the group consisting of zfr, smad2, spin1, and nipbl. According to some embodiments, the gene is selected from the group consisting of zfr, smad2, and spin1. According to some embodiments, the gene is selected from the group consisting of zfr and smad2. According to some embodiments, the gene is selected from the group consisting of smad2 and spin1. According to some embodiments, the gene is selected from the group consisting of smad2, spin1, and nipbl.

According to some embodiments, the gene encodes Zinc Finger RNA Binding Protein (ZFR).

The zfr gene (Gene ID 427424, synonym: zfr2) is conserved in a variety of animals including human, chimpanzee, dog, cow, mouse, and chicken. This gene encodes an RNA-binding protein characterized by its DZF (domain associated with zinc fingers) domain.

According to other embodiments, the gametolog is selected from the group consisting of smad2, st8sia3, kcmf1, spin1, sub1, chd1, nipbl, hnrnpk, gfbp1, mier3, btf3, golph3, vcp, txn11, nedd4, ctif, smad7, rpl17, znf532, hintz, c18orf25, atp5a, zswim6, rasa1, ube2r2, ubap2, and tcf4.

The gene smad2 encodes to the protein SMAD2 (e.g. Gene ID: 395247 in Gallus gallus (chicken)), also named SMAD family member 2 (Mothers against decapentaplegic homolog 2) SMAD2 protein mediates the signal of the transforming growth factor (TGF)-beta.

The gene st8sia3 encodes for st8sia3 protein (ST8 alpha-N-acetyl-neuraminide alpha-2,8-sialyltransferase 3; e.g. Gene ID: 414796 (Gallus gallus)).

The gene kcmf1 encodes for potassium channel modulatory factor 1 (e.g. Gene ID: 770239 (Gallus gallus)).

The gene spin1 encodes for SPIN1, a spindlin 1 protein (e.g. Gene ID: 395344 10 (Gallus gallus)).

The gene sub1 encodes for SUB1, regulator of transcription (e.g. Gene ID: 427425 (Gallus gallus)).

The gene chd1 encodes for CHD1 protein, a chromodomain helicase DNA binding protein 1Z (e.g. Gene ID: 395783 (Gallus gallus)).

The gene nipbl or LOC427439 encodes for Nipped-B homolog-like protein (e.g. Gene ID: 427439 (Gallus gallus)).

The gene hnrnpk encodes for HNRNPK, a heterogeneous nuclear ribonucleoprotein K (e.g. Gene ID: 427458 (Gallus gallus)).

The gene mier3 encodes for MIER3 or MIER family member 3 (e.g. Gene ID: 427146 (Gallus gallus)).

The gene golph3 encodes GOLPH3, golgi phosphoprotein 3 (e.g. Gene ID: 427422 (Gallus gallus)).

The gene vcp encodes VCP, a valosin containing protein (e.g. Gene ID: 427410 (Gallus gallus)).

The gene txn11 encodes TXNL1, a thioredoxin like 1 protein (e.g. Gene ID: 426854 (Gallus gallus)).

The gene ctif encodes CTIF, a CBP80/20-dependent translation initiation factor (e.g. Gene ID: 770140 (Gallus gallus)).

The gene smad7 encodes SMAD7 or SMAD family member 7 protein (Gene ID: 429683 (e.g. Gallus gallus)).

The gene rpl17 encodes ribosomal protein L17 (e.g. Gene ID: 426845 (Gallus gallus)).

The gene znf532 encodes for zinc finger protein 532 (e.g. Gene ID: 100857356 (Gallus gallus)).

The gene c18orf25 or LOC100858742 encodes chromosome Z open reading frame, human C18orf25 pseudogene (e.g. Gene ID: 100858742 (Gallus gallus)).

The gene zswin16 encodes a zinc finger SWIM-type containing 6 (e.g. Gene ID: 770670 (Gallus gallus)).

The gene rasa1 encodes for RASA1, a RAS p21 protein activator 1 (e.g. Gene ID: 427327 (Gallus gallus)).

The gene ube2r2 encodes a ubiquitin conjugating enzyme E2 R2 (e.g. Gene ID: 427021 (Gallus gallus)).

The gene ubap2 encodes for UBAP2, a ubiquitin associated protein 2 (e.g. Gene ID: 407092 (Gallus gallus)).

The gene tcf4 encodes for TCF4, a transcription factor 4 (e.g. Gene ID: 768612 (Gallus gallus)).

It is to be understood that the above gametologs of Gallus gallus (chicken) are given as non-limiting examples of gametologs, which includes their homologues in chickens, quails and other bird species as disclosed herein.

The term “meiosis-associated gene” as used herein refers to a gene encoding a product that is involved in the meiosis process.

According to some embodiments, the cell is a primordial germ cell (PGC). According to some embodiments, the PGC is selected from the group consisting of gonadal PGC, blood PGC and germinal crescent PGC. According to additional embodiments, the cell is selected from the group consisting of gonadal PGC, blood PGC and germinal crescent PGC. In another embodiment, the cell is a spermatogonial stem cell (SSC). In other embodiments, the cell is a spermatogonium or a spermatocyte. In another embodiment, the cell is a gamete (e.g. sperm cell).

Primordial germ cells are diploid cells that are precursors of gametes, and which still have to reach the gonads and there, following meiosis, are developed as haploid sperm and eggs. These cells can be obtained from embryos and be propagated as a cell culture without losing the ability to contribute to the germline when reintroduced into a host bird animal. PGCs can be genetically modified in culture using traditional transfection and selection techniques, including gene targeting and site-specific nuclease approaches.

According to some embodiments, a bird having the at least one cell is provided.

According to some embodiments, the bird is a chimeric bird. According to certain embodiments, the bird is a chimeric male bird having at least one PGC as described herein. According to certain embodiments, the at least one PGC is heterozygous to the genetically edited chromosome Z.

According to some embodiments, the bird is a female bird. According to some embodiments, the bird is a female bird having at least one PGC as described herein.

As used herein, the term “bird” refers to any avian species, including but not limited to chicken, quail, turkey, and duck. Preferably, the bird is a poultry.

According to some embodiments, the bird is a chicken. According to certain embodiments, the bird is a quail.

According to some embodiments, there is provided a cell population comprising the at least one cell. According to certain embodiments, the cell population comprises gametes.

According to some embodiments of the invention, the cell population are derived from the same avian species as the recipient bird. According to some embodiments of the invention, the cell population is derived from the same breed as the recipient bird. According to other embodiments, the cell population is derived from a different avian species or breed as the recipient bird.

According to an additional aspect, the present invention provides a site-directed mutagenesis system for reducing the expression and/or activity of at least one chromosome Z-gametolog.

Any genetically modification, editing or mutagenesis method known in the art that will result in the disruption of chromosome Z-gametolog expression or activity may be used according to the present invention.

According to some embodiments, the site-directed mutagenesis system is Clustered Regularly Interspaced Short Palindromic Repeats (CRISPR).

According to some embodiments, the CRISPR system comprises, or encodes: (i) a gRNA as described herein and (ii) an RNA-guided DNA endonuclease enzyme.

According to an additional aspect, the present invention provides a synthetic guide RNA comprising a nucleotide sequence (also referred to herein as a targeting nucleotide sequence) complementary to a target nucleic acid sequence within a bird chromosome Z-gametolog.

As used herein, “gRNA” means guide RNA and is a short synthetic RNA composed of a “scaffold” sequence necessary for endonuclease-binding and a user-defined nucleotide “spacer” or “targeting” sequence of approximately 20 nucleotides in length that defines the genomic target.

The gRNA molecule can be stabilized using modifications. According to some embodiments, the gRNA is a synthetic RNA molecule. According to some embodiments, the gRNA molecule is modified. According to certain embodiments, the gRNA is modified at the 5′ end.

In some embodiments, the modifications are selected from the group consisting of 2′-O-Methyl (2′-O-Me), 2′-O-methoxyethyl (2′-MOE), and combinations thereof.

The gRNA sequence includes a combination of a targeting homologous sequence (crRNA) and an endogenous bacterial RNA that links the crRNA to the Cas9 nuclease (tracrRNA) in a single chimeric transcript. The gRNA/Cas9 complex is recruited to the target sequence by the base-pairing between the crRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break.

According to some embodiments, the target nucleic acid sequence of the gRNA is within the coding region of the gametolog.

According to some embodiments, a nucleic acid construct encoding the guide RNA is provided.

According to some embodiments, a vector comprising at least one nucleic acid as described herein is provided. According to certain embodiments, the vector is a viral vector. According to certain embodiments, the viral vector is of a lentivirus or adenovirus.

The vectors typically comprise regulatory elements for the expression of the desired nucleic acids in the cells. The vector may comprise a promoter(s) which is operatively linked to drive the expression of the gRNA and the endonuclease. The promoter can be constitutive or inducible. According to some embodiments the promoter(s) operatively linked to drive the expression of the gRNA and the endonuclease are constitutive promoters. The promoter can be, but are not limited to, of a viral origin, such as the CMV, E1A, CAG or RSV promoter, or alternatively, a housekeeping promoter of the bird. According to certain exemplary embodiments, the gRNA promoter is 7SK promoter of quails. According to some embodiment, the gRNA promoter is human U6 promoter.

The CAG promoter is a strong synthetic promoter comprising CMV promoter and chicken beta-actin promoter frequently used to drive high levels of gene expression in birds.

According to some embodiments, the vectors further comprise functional element such as origin of replication, a multicloning site, and a selectable marker.

Preferably, the codons encoding the endonuclease of the DNA editing system are “optimized” codons, i.e., the codons are those that appear frequently in expressed genes in the bird species.

The present invention further provides an engineered, non-naturally occurring Clustered Regularly Interspaced Short Palindromic Repeats (CRISPR) gene-editing system comprising: (i) a synthetic guide RNA as described herein; and (ii) an RNA-guided DNA endonuclease enzyme.

According to some embodiments, the endonuclease is selected from the group consisting of caspase 9 (Cas9), Cpf1, zinc-finger nucleases (ZFNs), and transcription activator-like effector nucleases (TALENs).

As used herein, “Cas9” means non-specific CRISPR-associated endonuclease. The Cas9 nuclease has two functional domains: RuvC and HNH, each cutting a different DNA strand. When both of these domains are active, the Cas9 causes double strand breaks in the genomic DNA.

Cpf1 (CRISPR-Cas12a) is an endonuclease that uses a small guide RNA devoid of trans-activating CRISPR RNA, targets T-rich regions of the genome, and is able to generate double strand breaks (DSB) with staggered ends.

Zinc-finger nucleases (ZFNs) are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes.

Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). They contain DNA binding proteins called TALEs. The TALE is 33-35 amino acids in length and recognizes a single base pair of DNA.

According to some embodiments, the CRISPR editing system comprises a first nucleic acid sequence encoding the synthetic guide RNA and a second nucleic acid sequence encoding the RNA-guided DNA endonuclease enzyme. According to certain embodiments, the first and the second nucleic acid sequences each form a separate molecule. According to additional embodiments, the first and the second nucleic acid sequences are comprised in a single molecule.

According to some embodiments, a vector comprising the at least one engineered non-naturally occurring gene-editing system is provided. According to some embodiments, the vector is a viral vector. According to certain exemplary embodiments, the viral vector is lentivirus.

According to some embodiments, the invention relates to a nucleic acid molecule, construct, system or vector as disclosed herein, which modulates the expression of at least one Z-gametolog.

According to some embodiments, a cell population comprising the gene-editing system is provided.

According to some embodiments, a bird comprising at least one cell comprising the gene-editing system is provided. According to certain embodiments, the at least one cell is PGC. According to additional embodiments, the cell is selected from the group consisting of gonadal PGC, blood PGC and germinal crescent PGC. In another embodiment, the cell is a spermatogonial stem cell (SSC). In other embodiments, the cell is a spermatogonium or a spermatocyte. In another embodiment, the cell is a gamete (e.g. sperm cell).

In some embodiments, the cells are extracted form a bird embryo and the site-directed mutagenesis system is administered to the cells in vitro. In other embodiments, the site-directed mutagenesis system is administered to the bird or the embryo. In certain exemplary embodiments, the site-directed mutagenesis system is administered to the testicles of a sexually mature male bird. In other embodiments, the site-directed mutagenesis system is administered to a hatched chick before sexual maturation.

Any method as known in the art can be applied for administering the site-directed mutagenesis system, e.g. CRISPR, to the cells.

According to some embodiments, the site-directed mutagenesis system is administered to the cells using a viral vector. According to some embodiments, the viral vector is adenovirus. According to certain embodiments, the viral vector is lentivirus.

According to some embodiments, the site-directed mutagenesis system is administered to the cells using electroporation, a chemical agent, or nano particles.

According to some embodiments the chromosome Z-gametolog is mutated using the transposase system.

The transposase system comprises the transposase enzyme and a DNA element defined by its inverted terminal repeats (ITR) or other elements with the same ITRs. An example of transposase system is the Tol2 transposon. The transposase system enables the insertion of a DNA segment into a pre-defined location within the genome, thus the disruption of a desired gene.

Any site-directed mutagenesis can be used for generating the genetically modified birds described herein. An exemplary system is the Clustered Regularly Interspaced Short Palindromic Repeats (CRISPR) gene-editing system. The CRISPR system enables the cutting of strands of DNA in a precise location within the genome.

The CRISPR system uses a guide RNA (gRNA) to target the endonuclease to cut and create specific double-stranded breaks at a desired location(s) in the genome. The cleavage in the chromosome is then repaired by the error-prone non-homologous end joining (NHEJ) pathway. This pathway frequently causes small nucleotide insertions or deletions, which likely account for genetic disruption and gene knockout. This system is used herein to reduce the expression and/or activity of at least one chromosome Z-gametolog.

The targeting sequences are selected such that they will specifically hybridized to the gametolog sequences and not to any other chromosome of the cell.

Determining a suitable gRNA target sequence can be done using a variety of publicly available bioinformatic tools including the CHOPCHOP algorithm, Broad Institute GPP, CasOFFinder, CRISPOR, Deskgen, etc.

According to certain exemplary embodiments, the synthetic guide RNA comprises a targeting sequence selected from the group consisting of GGCTAGCTACACTGTCCACC (SEQ ID NO: 1) and GCGCACACAGCTACAGATTA (SEQ ID NO: 2).

It is to be understood, that when the nucleic acid sequence of a nucleic acid molecule of the invention is presented herein, both DNA and RNA sequences are included. For example, the sequence of a nucleic acid molecule having the nucleic acid sequence as set forth in SEQ ID NO: 1 may be either GGCTAGCTACACTGTCCACC or GGCUAGCUACACUGUCCACC, depending on the context.

Methods for qualifying the efficacy and detecting the correct genetically modifications as described herein are well known in the art and include, but not limited to, DNA sequencing, PCR, RT-PCR, RNase protection, in-situ hybridization, primer extension, Southern blot, Northern Blot and dot blot analysis.

The genetically editing or modifying systems of the invention may be used for the generation of male birds (e.g. roosters) having chromosome Z-gametolog with reduced activity and/or expression. The genetically edited male birds may be mated with females to generate female chickens that are capable of producing only viable female offspring.

As a first step, the DNA editing system is introduced into either primordial germ cells of the bird or directly into sperm cells (and/or precursors thereof as disclosed herein) of the bird. Any method know in the art can be used for introducing the DNA editing system including but not limited to, lipofection, transfection, microinjection, and electroporation, as well as transduction via viral vectors.

The cells are then screened in embodiments of the invention for those having chromosome Z-gametolog with reduced activity and/or expression.

To produce chimeric birds from PGCs edited in vitro, the exogenous edited cells are injected intravenously into surrogate host embryos, at a stage when their endogenous PGCs are migrating to the genital ridge.

Administration of the primordial germ cells to the recipient animal in-ovo can be carried out at any suitable time at which the PGCs can still migrate to the developing gonads. In one embodiment, administration is carried out from about stage IX according to the Eyal-Giladi & Kochav (EG&K) staging system to about stage 30 according to the Hamburger & Hamilton staging system of embryonic development, and in another embodiment, at stage 15. For chickens, the time of administration is thus during days 1, 2, 3, or 4 of embryonic development: in one embodiment day 2 to day 2.5. Administration is typically by injection into any suitable target site, such as the region defined by the amnion (including the embryo), the yolk sac, etc. According to some embodiments, the injection is into the embryo itself (including the embryo body wall), and in alternative embodiments, intravascular or intracoelomic injection into the embryo can be employed. In other embodiments, the injection is performed into the heart. The methods of the presently disclosed subject matter can be carried out with prior sterilization of the recipient bird in ovo (e.g. by chemical treatment using Busulfan of by gamma or X-ray irradiation). As used herein, the term “sterilization” refers to render partially or completely incapable of producing gametes derived from endogenous PGCs. When donor gametes are collected from such a recipient, they can be collected as a mixture with gametes of the donor and the recipient. This mixture can be used directly, or the mixture can be further processed to enrich the proportion of donor gametes therein.

The in-ovo administration of the primordial germ cells can be carried out by any suitable technique, either manually or in an automated manner. According to some embodiments, the in-ovo administration is performed by injection. The mechanism of in-ovo administration is not critical, but it is understood that the mechanism should not unduly damage the tissues and organs of the embryo or the extraembryonic membranes surrounding it so that the treatment will not unduly decrease hatch rate. A hypodermic syringe fitted with a needle of about 18 to 26 gauge is suitable for the purpose. A sharpened pulled glass pipette with an opening of about 20-50 microns diameter may be used. Depending on the precise stage of development and position of the embryo, a one-inch needle will terminate either in the fluid above the chick or in the chick itself. If desired, the egg can be sealed with a substantially bacteria-impermeable sealing material such as wax or the like to prevent subsequent entry of undesirable bacteria. It is envisioned that a high-speed injection system for avian embryos will be particularly suitable for practicing the presently disclosed subject matter. All such devices, as adapted for practicing the methods disclosed herein, comprise an injector containing a formulation of the primordial germ cells as described herein, with the injector positioned to inject an egg carried by the apparatus in the appropriate location within the egg. In addition, a sealing apparatus operatively connected to the injection apparatus can be provided for sealing the hole in the egg after injection thereof. According to other embodiments, a pulled glass micropipette can be used to introduce the PGCs into the appropriate location within the egg—for example directly into the blood stream, either to a vein or an artery or directly into the heart.

The injected embryo may be allowed to grow to maturity. In some embodiments, the injected embryo is transferred to a surrogate egg.

Once the eggs have been injected with the modified PGCs, the chimeric embryo is incubated to hatch. It is raised to sexual maturity, wherein the chimeric bird produces gametes derived from the donor PGCs.

The gametes, (either eggs or sperm) from the chimeras are then used to raise founder birds (e.g. chickens). Molecular biology techniques known in the art (e.g. PCR, Southern blot and/or T7 endonuclease assay) may be used to confirm germ-line transmission.

According to other embodiments, a genetic manipulation, in which site directed mutagenesis is applied, is performed directly on spermatogonial stem cells (SSCs) or differentiated sperm cells of a sexually mature male bird. This can be done by injecting or otherwise applying the site directed mutagenesis system described herein directly into its testicles.

According to additional embodiments, the site directed mutagenesis system described herein is injected or otherwise applied to testicles of non-mature birds, or chicks.

According to some embodiments, the genetically modified PGC cells described herein are administered to a male bird. In some embodiments, the PGC cells are administered to the testicles of the bird. In some embodiments, the birds are sexually mature. According to other embodiments, the birds are non-sexually mature, or chicks.

According to some embodiments, birds are sterilized before the administration.

In some embodiments, the mutagenesis system is administered using a viral vector, such as of lentivirus. In additional embodiments, the mutagenesis system is administered using transposases.

According to some embodiments, a lentivirus vector is used for delivering the site directed mutagenesis. In some embodiments, the site-directed mutagenesis is CRISPR. According to some embodiments, the lentivirus comprises both gRNA comprising targeting sequence to a Z-gametolog and a sequence encoding an endonuclease. According to some embodiments, the endonuclease is CAS9. According to certain embodiments, the lentivirus vector comprises a CAG promoter operably linked to the sequence encoding the endonuclease and/or the gRNA. According to certain embodiments, the endonuclease comprises a nuclear localization signal.

According to an additional aspect, there is provided a chimeric male bird having cells comprising at least one chromosome Z-gametolog having reduced expression and/or activity and an unmodified chromosome Z.

According to some embodiments, the bird does not comprise any exogenous polynucleotide sequence stably integrated into its genome.

The present invention provides methods of generating a chimeric male bird having cells comprising at least one chromosome Z-gametolog having reduced expression and/or activity and an unmodified chromosome Z, the method comprising the step of applying the site-directed mutagenesis system or the gene-editing system as described herein to a population of male bird cells, thereby generating genome-edited bird cells; and transferring the genome-edited bird cells to a recipient male bird embryo, thereby generating the chimeric male bird.

According to some embodiments, the method comprises raising the chimeric bird to sexual maturity, wherein the chimeric bird produces gametes derived from the donor's genetically modified PGCs.

The present invention further provides a method of generating a chimeric male bird having cells comprising at least one chromosome Z-gametolog having reduced expression and/or activity and an unmodified chromosome Z, the method comprising the step of administering the site-directed mutagenesis system or the gene-editing system as described herein to a recipient male bird embryo.

The chimeric bird is then mated with a female bird to generate heterozygous ZZ* offspring.

According to some embodiments, there is provided a method for generating the genetically edited male bird comprising the step of breeding a chimeric male bird as described herein with a female bird having unmodified chromosome Z. According to certain embodiments, the method comprises screening the resulting offspring for heterozygous ZZ* birds.

According to an additional aspect the present invention provides a genetically edited female bird capable of laying viable egg population with biased sex ratio, said bird having a reduced expression and/or activity of at least one chromosome Z-gametolog.

According to some embodiments, there is provided a method for generating the genetically edited female bird capable of laying viable egg population with biased sex ratio, comprising the step of crossing the genetically edited male bird described herein with a female bird and screening the offspring for genetically edited females.

According to an additional aspect, the present invention provides a method for producing a bird hatchling population characterized by a sex ratio biased towards females, comprising breeding the genetically edited female bird as described herein with a male bird having unmodified Z-chromosome, thereby producing an essentially female-only hatchling population.

According to an additional aspect, the present invention provides a veterinary composition comprising the PGCs cells or the site-directed mutagenesis system as described herein and an acceptable carrier.

According to some embodiments, the veterinary composition is formulated for injection to birds.

According to some embodiments, the site directed mutagenesis system is CRISPR.

According to some embodiments, the composition comprises a viral vector or transposase comprising the site directed mutagenesis system described herein.

According to some embodiments, the composition further comprises antibiotics.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1: Editing Zfr Gene Using CRISPR System

Bioinformatics Analysis for Guide RNAs (gRNAs) Selection:

Focusing on the 3^{rd} exon of the zfr gene from Gallus gallus's Z chromosome, 3 gRNA were selected. The selected targeting sequences were further analyzed using CHOPCHOP algorithm (Labun, K. et al. Nucleic Acids Res. 47, W171-W174 (2019)) before testing their efficiency in vitro. DNA sequence of ˜1000 bp upstream to the exon, the 283 bp of the exon itself and ˜1000 bp downstream to the exon were inserted as a single target sequence to the CHOPCHOP analysis with the following parameters: comparison genome of Gallus gallus 6 (galGal6), using CRISPR/Cas9, for knock-out.

The 2 gRNA targeting sequences described below and their information were located within the analysis report.

Cas9 in-vitro cleavage assay (Anders, C. & Jinek, M. Methods in Enzymology 546, 1-20 (Elsevier Inc., 2014)):

gRNAs were chosen for targeting of zfr gene from the Z chromosome of Gallus gallus. The 2 gRNAs comprising the targeting sequences SEQ ID NO: 1 and SEQ ID NO: 2 were synthesized in-vitro, and underwent cleavage assessment using a PCR product of the zfr DNA target sequence and purified Cas9 endonuclease protein. DNA product cleavage was analyzed on an agarose gel.

### In-Vivo Cleavage Assay:

In-vivo assay was done utilizing Mashiko et. al. pEGxxFP construct (RNA. Sci. Rep. 3, 3355 (2013)). The target sequence comprised of partial zfr gene from the Z chromosome of Gallus gallus that was cloned in between overlapping segments of EGFP gene. The construct was transfected into chicken Fibroblast (DF-1) or human embryonic kidney 293 cells (HEK) and observed for green fluorescence after ˜72 hrs.

### Results:

CHOPCHOP analysis report for the target area inside the zfr gene at the Z chromosome, resulted in 192 possible gRNAs sorted from best to worse. The 3 chosen gRNAs were located in the report as follows: gRNA 1 (having targeting sequence SEQ ID NO: 1) ranked 17^{th}, gRNA 2 ranked 113^{th} and gRNA 3 (having targeting sequence SEQ ID NO: 2) ranked 7^{th} (Table 1). Apart from the gRNAs rank, the number of off-target sites that exist in the Gallus gallus genome was also a significant consideration. The higher the number of off-targets, the less optimal the gRNA. gRNA 2 has considerably more off-targets than gRNAs 1 and 3 (Table 1). Moreover, one of the off-targets has 0 mismatches and matches the target sequence on 100% (confirmed as an off-target located at the W chromosome zfr gene). Thus, gRNA 2 is considered as a poor choice for actual usage. Regarding gRNAs 1 (having targeting sequence SEQ ID NO: 1) and 3 (having targeting sequence SEQ ID NO: 2) off-targets (Table 2), each gRNA has an off-target sequence with 1 mismatch at the W chromosome ZFR gene. Additionally, gRNA 1 has a second off-target site at the 1^{st} chromosome with 3 sequence mismatches. Overall gRNAs 1 and 3 mismatches are considered as a reasonable result, especially when taking into account the homology between the zfr genes from the W or Z chromosomes. Hence, gRNAs 1 and 3 (having targeting sequences as set forth in SEQ ID NOs: 1 and 2, respectively) were used for further analysis.

TABLE 1
CHOPCHOP ranking result for the 3 chosen gRNAs. MMX
meaning the number of Gallus gallus genomic off-
targets with X mismatches from the target sequence.

| Chosen | CHOPCHOP rank | | | | | |
|---|---|---|---|---|---|---|
| gRNA | (out of 192) | MM0 | MM1 | MM2 | MM3 | Efficiency |
| 1 | 17 | 0 | 1 | 0 | 1 | 58.45 |
| 2 | 113 | 1 | 0 | 0 | 185 | 40.52 |
| 3 | 7 | 0 | 1 | 0 | 0 | 43.71 |

TABLE 2
Detailed off-targets for gRNAs 1 and 3
according to CHOPCHOP.

| | | |
|---|---|---|
| | | Targeting sequence |
| | Number of | (including |
| Location | mismatches | mismatches) |

gRNA 1 off-targets

| | | |
|---|---|---|
| Chr 1: | 3 | CCTGGTGaAgAGgGTAGCTAGCC |
| 31573888 | | |
| Chr W: | 1 | CCTGGTGGACAGTGTAGCTAGCa |
| 5189123 | | |

gRNA 3 off-targets

| | | |
|---|---|---|
| Chr W: | 1 | CCATAATCTGTAGCTGcGTGCGC |
| 5188972 | | |

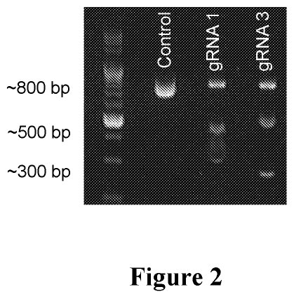

Initial gRNAs targeting and cleavage testing were performed using a Cas9 in-vitro cleavage assay (Anders ibid). FIG. 2 presents digestion patterns of the target DNA sequence using gRNA 1 or gRNA 3 compared to non-digested target DNA of 768 bp. The cleavage pattern for gRNA 1 shows that while some of the target DNA remained uncut, two smaller bands at ˜300 bp and ˜450 bp were apparent and matched the cleavage prediction of gRNA 1 on the target sequence. The gRNA 3 cleavage pattern suggest that it also contained some uncut target DNA and two smaller bands corresponding to ˜280 bp and ˜480 bp that matched the cleavage prediction of gRNA 3. Hence in-vitro assay for both gRNA 1 and gRNA 3 demonstrated positive cleavage.

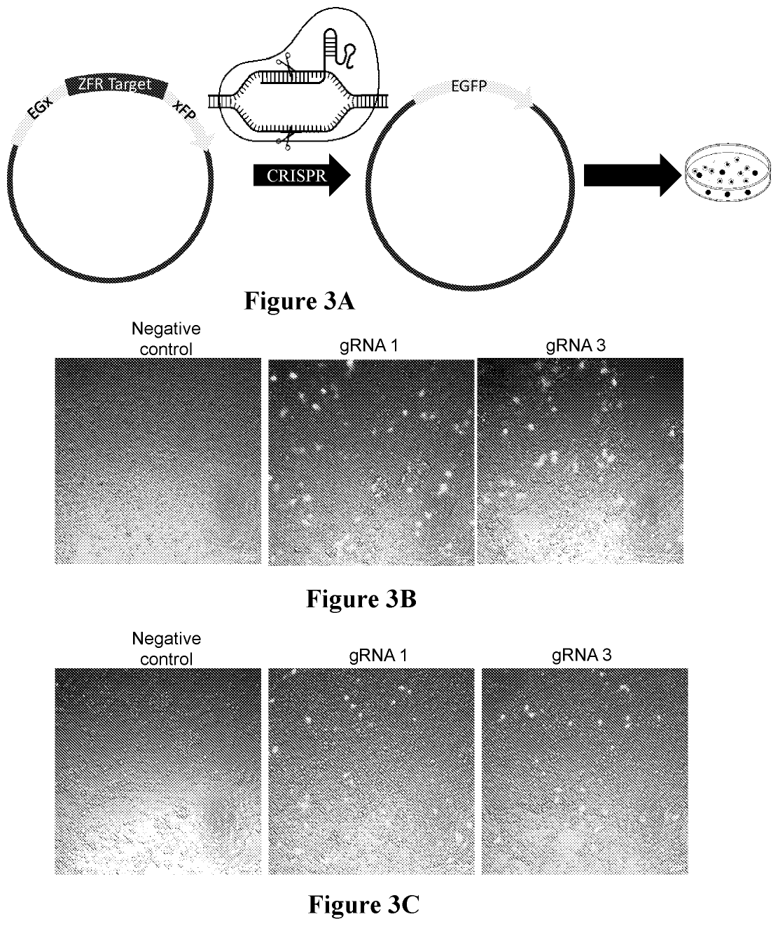

An in-vivo assay was also performed to examine the activity of the selected gRNA molecules. The in-vivo assay, despite not testing cleavage ability on the chromosome itself, provided a more reliable representation on the gRNAs cleavage potential in a complex cellular environment. Using a pEGxxFP construct (Mashiko, ibid), containing the target sequence of zfr in between overlapping areas from EGFP reporter, the pEGxxFP zfr plasmid was co-transfected with a second plasmid containing gRNA (apart from the control experiment) and Cas9 endonuclease (FIG. 3a). The assay was carried out in HEK 293 cells (FIG. 3b) and chicken Fibroblast cells (DF-1) (FIG. 3c), where a positive cleavage was aimed to result in a green fluorescence signal within the cell.

The in-vivo assay results clearly demonstrate the correct activity of the designed gRNA molecules (FIG. 3b, FIG. 3c). Control experiments, in the absence of a gRNA, did not develop green fluorescence. Thus, the pEGxxFP zfr construct is stable and does not cleave and self-repair spontaneously. A clear green signal was observed for cells co-transfected with the pEGxxFP zfr construct together with gRNA 1 or gRNA 3. As the control did not result in any background fluorescence, it is concluded that all fluorescence signals originated from gRNAs cleavage activity on the pEGxxFP zfr construct and EGFP repair. In terms of efficiency, gRNA 1 appeared to result in better fluorescence as green cells were more abundant than for gRNA 3, corresponding well to the predicted efficiency by the CHOPCHOP algorithm (Table 1). These results served as further indication for the two selected gRNAs ability to cleave the target sequence within the ZFR gene from a Gallus gallus's Z chromosome and demonstrate favoring gRNA 1 over gRNA 3.

### Example 2: Producing a Genetically Edited Female Bird That Is Capable of Producing Only Female Offspring

The DNA editing system described in Example 1 is used to knockout the expression of zfr in primordial germ cells (PGCs). The modified cells having ZZ* are then administered to a male chicken embryo. The administration is performed under conditions sufficient to allow the PGC cells to colonize a gonad of the recipient bird embryo. The embryo is raised to maturity. The chimeric bird is then mated with regular (native) females and the progeny are screened for heterozygote ZZ* birds. The identified heterozygous ZZ* are mated with native females ('Grandmothers' WZ), and their offspring are screened for female WZ* ('Mothers'). The genetically modified WZ* are the layer bird females that are capable of producing only female offspring. The resulting offspring are non-genetically modified birds.

### Example 3: Injection of the Site-Directed Mutagenesis System Into Sexually Mature Male Testes for Producing Chimeric Heterozygote ZZ* Birds

Lentiviral vectors comprising the DNA editing system as described in Example 1 were designed, suitable to be used to knock-out the expression of zfr in quails or roosters. The designed lentiviral vector comprised gRNA scaffold having promoter 7SK of quails, and Cas9 endonuclease having CAG promoter.

A surgical procedure was performed on hatched male quails as follows. Male quails at an age between 1-6 weeks were used. Under anesthesia, the first testis was exposed within the bird's body. Using a syringe, a suspension comprising Lentiviral vectors were injected into the testis at several locations. The surgical opening was sutured and closed. The same procedure was executed on the second testis from the other side of the bird. Following Lentivirus injection to both testes, the male was given 1-2 weeks of recovery. In other experiments, the procedure is repeated on 1-26 week-old roosters.

After recovery, the male (considered as GO) is put together with females to mate. Eggs are hatched for scanning transgenic offspring (G1) having *ZZ.

In an additional experiment a transposase system is used for delivering the site-mutagenesis system to the bird testicles. In this case the injected liquid contains a transfection reagent (such as lipofectamine), plasmid for expression of transposase and plasmid for desired genomic integration (i.e. disruption of the Z-gametolog).

In additional experiment, injection of primordial germ cells into male testes is performed. In this case the injected liquid contains modified PGCs (ZZ*). The PGCs are injected into a native male or to a male that was sterilized prior to the procedure (by e.g. utilizing radiation (UV/Gamma) or specific chemicals (like Busulfan)). Once sterilized, the surgical procedure to implant the new PGCs is performed as described above.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A male bird cell having at least one genetically modified chromosome Z, wherein the genetically modified chromosome comprises at least one chromosome Z-gametolog having reduced expression and/or activity, the bird cell is capable of developing into functional gametes.

2. The cell of claim 1, wherein said cell is genetically edited using at least one artificially engineered nuclease.

3. The cell of claim 1, wherein the gametolog is a gene selected from the group consisting of zfr, smad2, st8sia3, kcmf1, spin1, sub1, chd1, nipbl, hnrnpk, gfbp1, mier3, btf3, golph3, vcp, txn11, nedd4, ctif, smad7, rpl17, znf532, hintz, c18orf25, atp5a, zswim6, rasa1, ube2r2, ubap2, and tcf4.

4. (canceled)

5. (canceled)

6. (canceled)

7. The cell of claim 1, wherein at least one of the following holds true:
a. said cell is a primordial germ cell (PGC);
b. said bird is a poultry;
c. the bird is selected from the group consisting of chicken, quail, turkey, goose, and duck.

8. (canceled)

9. (canceled)

10. (canceled)

11. A cell population comprising at least one cell according to claim 1.

12. A bird having at least one cell according to claim 1.

13. A chimeric male bird having at least one PGC according to claim 7.

14. (canceled)

15. A synthetic guide RNA comprising a targeting nucleotide sequence complementary to a target nucleic acid sequence within a bird chromosome Z-gametolog or a site-directed mutagenesis system for reducing the expression and/or activity of at least one chromosome Z-gametolog.

16. (canceled)

17. The synthetic guide RNA of claim 15, wherein the Z-gametolog is a gene selected from the group consisting of zfr, smad2, st8sia3, kcmf1, spin1, sub1, chd1, nipbl, hnrnpk, gfbp1, mier3, btf3, golph3, vcp, txn11, nedd4, ctif, smad7, rpl17, znf532, hintz, c18orf25, atp5a, zswim6, rasa1, ube2r2, ubap2, and tcf4.

18. (canceled)

19. (canceled)

20. (canceled)

21. The synthetic guide RNA of claim 15, wherein at least one of the following holds true:
a. the target nucleic acid sequence is within exon 3 of zfr;
b. the synthetic guide RNA comprises a targeting sequence selected from the group consisting of SEQ ID NO: 1 and SEQ ID NO: 2.

22. (canceled)

23. (canceled)

24. A vector comprising at least one nucleic acid construct encoding the synthetic guide RNA of claim 15.

25. The vector of claim 24, wherein said vector is a viral vector.

26. (canceled)

27. (canceled)

28. An engineered, non-naturally occurring Clustered Regularly Interspaced Short Palindromic Repeats (CRISPR) gene-editing system comprising: (i) a synthetic guide RNA according to claim 15, and (ii) an RNA-guided DNA endonuclease enzyme.

29-34. (canceled)

35. A cell population comprising the gene-editing system of claim 28.

36. A bird comprising at least one cell comprising the gene-editing system of claim 28.

37. The bird of claim 36, wherein the at least one cell is a PGC.

38. A chimeric male bird having cells according to claim 1, wherein the cells comprise a genetically modified chromosome Z comprising at least one chromosome Z-gametolog having reduced expression and/or activity and an unmodified chromosome Z.

39. (canceled)

40. (canceled)

41. A method of generating a chimeric male bird according to claim 38, the method comprising the step of
a. applying a site-directed mutagenesis system for reducing the expression and/or activity of at least one chromosome Z-gametolog, or an engineered, non-naturally occurring Clustered Regularly Interspaced Short Palindromic Repeats (CRISPR) gene-editing system comprising a targeting nucleotide sequence complementary to a target nucleic acid sequence within a bird chromosome Z-gametolog to a population of male bird cells, thereby generating genome-edited bird cells; and transferring the genome-edited bird cells to a recipient male bird embryo, thereby generating the chimeric male bird;
b. administering a site-directed mutagenesis system for reducing the expression and/or activity of at least one chromosome Z-gametolog, or an engineered, non-naturally occurring CRISPR gene-editing system, comprising a targeting nucleotide sequence complementary to a target nucleic acid sequence within a bird chromosome Z-gametolog to a recipient male bird embryo;
c. administering (i) a PGC having at least one genetically modified chromosome Z, wherein the genetically modified chromosome comprises at least one chromosome Z-gametolog having reduced expression and/or activity wherein the bird cell is capable of developing into functional gametes, (ii) a site-directed mutagenesis system for reducing the expression and/or activity of at least one chromosome Z-gametolog, or (iii) an engineered, non-naturally occurring CRISPR gene-editing system comprising a targeting nucleotide sequence complementary to a target nucleic acid sequence within a bird chromosome Z-gametolog, to the testicles of a recipient male bird; and/or administering a viral vector comprising a site-directed mutagenesis system for reducing the expression and/or activity of at least one chromosome Z-gametolog, or an engineered, non-naturally occurring CRISPR gene-editing system comprising a targeting nucleotide sequence complementary to a target nucleic acid sequence within a bird chromosome Z-gametolog, to the testicles of a recipient male bird; or
d. breeding a chimeric male bird having cells comprising a genetically modified chromosome Z comprising at least one chromosome Z-gametolog having reduced expression and/or activity and an unmodified chromosome Z, with a female bird having unmodified chromosome Z and screening the resulting offspring for genetically modified males.

42.-48. (canceled)

49. A genetically modified female bird capable of laying viable egg population with biased sex ratio, said bird having a reduced expression and/or activity of at least one chromosome Z-gametolog.

50. A method of generating a genetically modified female bird capable of laying viable egg population with biased sex ratio, comprising crossing the chimeric male bird of claim 38 with a female bird and screening the offspring for genetically modified females.

51. A method for producing a bird hatchling population characterized by a sex ratio biased towards females, comprising breeding the genetically modified female bird of claim 49 with a male bird having unmodified Z-chromosomes, thereby producing an essentially female-only hatchling population.


SEQUENCE LISTING (2 sequences)

SEQ ID NO: 1 - DNA, 20 bp, Artificial sequence
  Guide RNA
ggctagctacactgtccacc

SEQ ID NO: 2 - DNA, 20 bp, Artificial sequence
  Guide RNA
gcgcacacagctacagatta